(19) United States
(12) Patent Application Publication
(10) Pub. No.: US 2024/0398773 A1
(43) Pub. Date: Dec. 5, 2024

(54) COMPOSITIONS COMPRISING OTESECONAZOLE

(71) Applicant: Mycovia Pharmaceuticals, Inc., Durham, NC (US)

(72) Inventors: Alexander SMITH, Apex, NC (US); Mark COFFIN, New Hill, NC (US)

(21) Appl. No.: 18/580,864

(22) PCT Filed: Jul. 22, 2021
(86) PCT No.: PCT/US2021/042691

§ 371 Date: Jan. 19, 2024

(51) Int. Cl. A61K 31/4439; A61K 9/20; A61K 9/48

(52) U.S. Cl. CPC A61K 31/4439; A61K 9/2013; A61K 9/2054; A61K 9/4858; A61K 9/4866

(57) ABSTRACT

The present application relates to capsules and tablets comprising 150 mg or 300 mg oteseconazole, a method for preparing them, and their use for the treatment or prevention of RVVC, inflammatory bowel disease, psoriasis, systemic fungal infection, skin structure fungal infection, mucosal fungal infection, or onychomycosis.

This application is a National Stage application of International Application No. PCT/US2021/042691, filed July 22, 2021.

### TECHNICAL FIELD

The present application relates to a pharmaceutical composition comprising oteseconazole according to compound 1. In particular, the present application seeks to provide a method for treating or preventing vulvovaginal candidiasis using compound 1.

### BACKGROUND OF THE INVENTION

WO 2011133875 (Applicant: Viamet Pahrmaceuticals. Inc.) describes oteseconazole active substance and similar metalloenzyme inhibitor compounds. Oteseconazole has been developed as an oral fungal inhibitor to treat a range of fungal pathogens. For example, oteseconazole is being developed as treatment for recurrent vulvovaginal candidiasis (RVVC), a debilitating chronic condition that affects millions of women worldwide. While oral administration is the most preferred and patient friendly route for administration, oral delivery of oteseconazole at a dosage that is effective to treat acute VVC episodes in women with RVVC remains a challenge. Accordingly, research continues into the development of oteseconazole oral delivery that can provide for practical unit oral dosage forms for the treatment of RVVC. The present application relates to such unit oral dosage suitable for the treatment of RVVC.

### BRIEF SUMMARY

The present application is directed to a composition comprising compound 1 (also known as oteseconazole and VT-1161), or a pharmaceutically acceptable salt thereof:

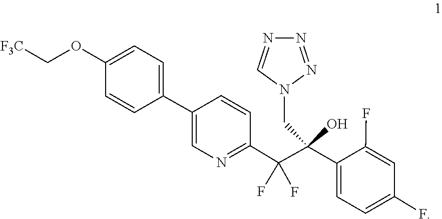 [SMILES: O[C@@](Cn1cnnn1)(c1ccc(F)cc1F)C(F)(F)c1ccc(-c2ccc(OCC(F)(F)F)cc2)cn1]

The composition comprises an anhydrous polymorph of oteseconazole, a binder, a filler, a disintegrant and a surfactant. Some embodiments may comprise a lubricant. Oteseconazole is useful for treating or preventing inflammatory bowel disease, psoriasis, systemic fungal infection, skin structure fungal infection, mucosal fungal infection, or onychomycosis, and RVVC. In some embodiments, the use or method comprises administering to a subject in need thereof a suitable unit oral dosage of oteseconazole in an amount effective to treat the condition. The present application is also directed to a method of manufacturing any of the compositions and embodiments described herein.

### DESCRIPTION OF DRAWINGS

FIG. 1: Manufacturing Process Flow Diagram for Oteseconazole Capsule, 150 mg (FIGS. 1A, 1B, and IC are continuation of the manufacturing process and together form FIG. 1).

FIG. 2: Mean Oteseconazole Plasma Concentration over Time: Cohorts 3-8.

FIG. 3: Mean Oteseconazole Plasma Concentration over Time.

FIG. 4: Mean Oteseconazole Plasma Concentration over Time (C1-011).

FIG. 5: Mean Oteseconazole Plasma Concentration over Time (CL-012).

FIG. 6: Mean Oteseconazole Plasma Concentration over Time-Intense PK Sampling.

FIG. 7: Mean Oteseconazole Plasma Concentration over Time.

### DETAILED DESCRIPTION

In some aspects, oteseconazole is administered in an oral dosage form as a solid or liquid formulation. Suitable solid dosage forms of oteseconazole include tablets, capsules, sachets, powders, granules, orally dispersible films, etc. Suitable liquid oral dosage forms of oteseconazole include syrups, solutions, ampoules, dispersions, semi-solids, softgels, etc. It would be understood that oteseconazole can be administered in an encapsulated liquid formulation such that while the dosage form is a solid form, the active is in a liquid form. Regardless of the form of administration, it is contemplated that any immediate release formulation of oteseconazole may be suitable in the unit dosages described herein.

In some embodiments, the pharmaceutical composition comprises an anhydrous polymorph of oteseconazole, a binder, a filler, a disintegrant and a surfactant and optionally a lubricant. Fillers suitable to the compositions of the present application include lactose, microcrystalline cellulose or calcium hydrogen phosphate. Binders suitable to the compositions of the present application include hydroxypropyl cellulose, pregelatinized maize starch, and polyvinylpyrrolidone. A suitable disintegrant may be croscarmellose sodium, potato starch and sodium starch glycolate. A suitable surfactant is sodium lauryl sulfate.

In some embodiments, the composition comprises about 150 mg oteseconazole, about 20 mg silicified microcrystalline cellulose, about 12 mg lactose monohydrate, about 6 mg hydroxypropyl cellulose, about 4 mg to about 8 mg croscarmellose sodium, about 2 mg sodium lauryl sulfate; and optionally about 2 mg magnesium stearate. In other embodiments, the composition comprises about 28% oteseconazole, about 20% silicified microcrystalline cellulose, about 42% lactose monohydrate, about 3% hydroxypropyl cellulose, about 2% to about 4% croscarmellose sodium, about 1% sodium lauryl sulfate; and optionally about 1% magnesium stearate. In further embodiments, the composition comprises about 75% oteseconazole, about 10% silicified microcrystalline cellulose, about 6% lactose monohydrate, about 3% hydroxypropyl cellulose, about 2% to about 4% croscarmellose sodium, about 1% sodium lauryl sulfate; and optionally about 1% magnesium stearate. In these embodiments, the composition may be formulated into a dosage unit comprising between about 150 mg and about 250 mg of the composition. In other embodiments, the total weight of the dosage unit is about 150 mg, about 180 mg, about 210 mg, or about 250 mg.

In some embodiments. The composition comprises about 300 mg oteseconazole, about 73 mg silicified microcrystalline cellulose, about 105 mg lactose monohydrate, about 16 mg hydroxypropyl cellulose, about 11 mg to about 22 mg croscarmellose sodium, about 5 mg sodium lauryl sulfate; and optionally about 5 mg magnesium stearate. In further embodiments, the composition comprises about 75% oteseconazole, about 10% silicified microcrystalline cellulose, about 6% lactose monohydrate, about 3% hydroxypropyl cellulose, about 2% to about 4% croscarmellose sodium, about 1% sodium lauryl sulfate; and optionally about 1% magnesium stearate. In a further embodiment, the composition comprises about 57% oteseconazole, about 14% silicified microcrystalline cellulose, about 20% lactose monohydrate, about 3% hydroxypropyl cellulose, about 2% to about 4% croscarmellose sodium, about 1% sodium lauryl sulfate; and optionally about 1% magnesium stearate. In certain embodiments, the composition comprises about 56% oteseconazole, about 14% silicified microcrystalline cellulose, about 20% lactose monohydrate, about 3% hydroxypropyl cellulose, about 2% to about 4% croscarmellose sodium, about 1% sodium lauryl sulfate; and optionally about 1% magnesium stearate. In further embodiments, the composition comprises about 67% oteseconazole, about 10% silicified microcrystalline cellulose, about 14% lactose monohydrate, about 3% hydroxypropyl cellulose, about 2% to about 4% croscarmellose sodium, about 1% sodium lauryl sulfate; and optionally about 1% magnesium stearate. In these embodiments, the composition may be formulated into a dosage unit comprising between about 400 mg and about 600 mg of the composition. In further embodiments, the total weight of the dosage unit comprising the composition is about 400 mg, about 440 mg, about 480 mg, about 520 mg, about 560 mg, or about 600 mg.

In some embodiments, the present application provides a pharmaceutical composition further comprising an additional therapeutic agent. In a further embodiment, the additional therapeutic agent is an anti-cancer agent, antifungal agent, cardiovascular agent, antiinflammatory agent, chemotherapeutic agent, an anti-angiogenesis agent, cytotoxic agent, an anti-proliferation agent, metabolic disease agent, ophthalmologic disease agent, central nervous system (CNS) disease agent, urologic disease agent, or gastrointestinal disease agent.

In one aspect, the present application provides a kit comprising an effective amount of oteseconazole, in unit dosage form, together with instructions for administering the compound to a subject suffering from or susceptible to vulvovaginal candidiasis or RVVC.

The term “pharmaceutically acceptable salt” or “pharmaceutically acceptable carrier” is meant to include salts of the active compounds, which are prepared with relatively nontoxic acids or bases, depending on the particular substituents found on the compounds described herein. When compounds of the present application contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present application contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydroiodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galacturonic acids and the like (see e.g., Berge et al., Journal of Pharmaceutical Science 66:1-19 (1977)). Certain specific compounds of the present application contain both basic and acidic functionalities that allow the compounds to be converted into either base or acid addition salts. Other pharmaceutically acceptable carriers known to those of skill in the art are suitable for the present application.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present application.

Oteseconazole and its pharmaceutically acceptable salts can exist in a variety of polymorphic solids, including unsolvated forms as well as solvated forms, including hydrated forms. In general, the solvated forms are equivalent to unsolvated forms and both forms are intended to be encompassed within the scope of the present application. Oteseconazole may exist in multiple crystalline or amorphous forms. In general, all physical forms are equivalent for the uses contemplated by the present application and are intended to be within the scope of the present application.

The present application also provides a pharmaceutical composition, comprising an effective amount of oteseconazole and a pharmaceutically acceptable carrier. Actual dosage levels and time course of administration of oteseconazole in the pharmaceutical compositions of the present application may be varied so as to obtain an amount of the active ingredient which is effective to achieve the desired therapeutic response for a particular patient, composition, and mode of administration, without being toxic (or unacceptably toxic) to the patient.

By “pharmaceutically effective amount” as used herein is meant an amount of oteseconazole high enough to significantly positively modify the condition to be treated but low enough to avoid serious side effects (at a reasonable benefit/risk ratio), within the scope of sound medical judgment. A pharmaceutically effective amount of oteseconazole for treating vulvovaginal candidiasis or RVVC will vary with the particular goal to be achieved, the age and physical condition of the patient being treated, the severity of the underlying disease, the duration of treatment, the nature of concurrent therapy and the specific dosage employed. For example, a therapeutically effective amount of oteseconazole administered to a child or a neonate will be reduced proportionately in accordance with sound medical judgment. The effective amount of oteseconazole will thus be the minimum amount, which will provide the desired effect.

The compound may be administered as a dispersion. Dispersions can also be prepared, for example, in glycerol, liquid polyethylene glycols, and mixtures thereof, and in oils.

Some examples of substances which can serve as pharmaceutical carriers are sugars, such as lactose, glucose and sucrose; starches such as corn starch and potato starch; cellulose and its derivatives such as sodium carboxymethylcellulose, ethylcellulose and cellulose acetates; powdered tragancanth; malt; gelatin; talc; stearic acids; magnesium stearate; calcium sulfate; vegetable oils, such as peanut oils, cotton seed oil, sesame oil, olive oil, corn oil and oil of theobroma; polyols such as propylene glycol, glycerin, sorbitol, mannitol, and polyethylene glycol; agar; alginic acids; pyrogen-free water; isotonic saline; and phosphate buffer solution; skim milk powder; as well as other non-toxic compatible substances used in pharmaceutical formulations such as Vitamin C, estrogen and echinacea, for example. Wetting agents and lubricants such as sodium lauryl sulfate, as well as coloring agents, flavoring agents, lubricants, excipients, tableting agents, stabilizers, anti-oxidants and preservatives, can also be present. Solubilizing agents, including for example, cremaphore and beta-cyclodextrins can also be used in the pharmaceutical compositions herein.

Pharmaceutical compositions comprising oteseconazole can be manufactured by means of conventional mixing, dissolving, granulating, dragee-making, levigating, emulsifying, encapsulating, entrapping or lyophilization processes. The compositions can be formulated in conventional manner using one or more physiologically acceptable carriers, diluents, excipients or auxiliaries which facilitate processing of the active compounds into preparations which can be used pharmaceutically.

The pharmaceutical compositions can take the form of, for example, lozenges, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets can be coated by methods well known in the art with, for example, sugars or enteric coatings.

Liquid preparations for oral administration can take the form of, for example, elixirs, solutions, syrups or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non aqueous vehicles (e.g., almond oil, oily esters, ethyl alcohol or fractionated vegetable oils); and preservatives (e.g., methyl or propyl p-hydroxybenzoates or sorbic acid). The preparations also can contain buffer salts, preservatives, flavoring, coloring and sweetening agents as appropriate.

The unit dosages may comprise oteseconazole, a pharmaceutically acceptable salt or a prodrug thereof.

The term “prodrug” includes compounds with moieties which can be metabolized in vivo. Generally, the prodrugs are metabolized in vivo by esterases or by other mechanisms to active drugs. Examples of prodrugs and their uses are well known in the art (See, e.g., Berge et al. (1977) “Pharmaceutical Salts”, J. Pharm. Sci. 66:1-19). The prodrugs can be prepared in situ during the final isolation and purification of the compounds, or by separately reacting the purified compound in its free acid form or hydroxyl with a suitable esterifying agent. Hydroxyl groups can be converted into esters via treatment with a carboxylic acid. Examples of prodrug moieties include substituted and unsubstituted, branch or unbranched lower alkyl ester moieties, (e.g., propionic acid esters), lower alkenyl esters, di-lower alkyl-amino lower-alkyl esters (e.g., dimethylaminoethyl ester), acylamino lower alkyl esters (e.g., acetyloxymethyl ester), acyloxy lower alkyl esters (e.g., pivaloyloxymethyl ester), aryl esters (phenyl ester), aryl-lower alkyl esters (e.g., benzyl ester), substituted (e.g., with methyl, halo, or methoxy substituents) aryl and aryl-lower alkyl esters, amides, lower-alkyl amides, di-lower alkyl amides, and hydroxy amides. Preferred prodrug moieties are propionic acid esters and acyl esters. Prodrugs which are converted to active forms through other mechanisms in vivo are also included. In aspects, the compounds of the invention are prodrugs of any of the formulae herein.

Particular prodrugs of oteseconazole suitable for the unit dosages of the present application include phosphate esters of the compound. In a phosphate ester of oteseconazole, the OH group of the active is replaced by the following group:

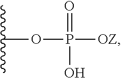

wherein Z may be an alkyl group (for mono-hydrogen phosphate esters) or H (for di-hydrogen phosphate ester).

The pharmaceutical compositions can, if desired, be presented in a pack or dispenser device which can contain one or more unit dosage forms containing the active compound(s). The pack can, for example, comprise metal or plastic foil, such as a blister pack. The pack or dispenser device can be accompanied by instructions for administration.

The pharmaceutically active component of the presently disclosed subject matter, or compositions thereof, will generally be used in an amount effective to treat inflammatory bowel disease, psoriasis, systemic fungal infection, skin structure fungal infection, mucosal fungal infection, or onychomycosis. In some embodiments, the pharmaceutically active component of the presently disclosed subject matter, or compositions thereof, will generally be used in an amount effective to treat or prevent vulvovaginal candidiasis or RVVC. The compound can be administered therapeutically to achieve therapeutic benefit or prophylactically to achieve prophylactic benefit. By therapeutic benefit is meant eradication or amelioration of vulvovaginal candidiasis or RVVC and/or eradication or amelioration of one or more of the symptoms associated with vulvovaginal candidiasis or RVVC such that the patient reports an improvement in feeling or condition, notwithstanding that the patient can still be afflicted with the underlying disorder.

For prophylactic administration, the compound can be administered to a patient at risk of infection of vulvovaginal candidiasis or developing RVVC. A patient at risk of developing a disease can be a patient having characteristics placing the patient in a designated group of at risk patients, as defined by an appropriate medical professional or group. A patient at risk may also be a patient that is commonly or routinely in a setting where development of the underlying disease that may be treated by administration of oteseconazole could occur. In other words, the at risk patient is one who is commonly or routinely exposed to vulvovaginal candidiasis or may be acutely exposed for a limited time. Alternatively, prophylactic administration can be applied to avoid the onset of symptoms in a patient diagnosed with the underlying disorder.

The amount of compound administered will depend upon a variety of factors, including, for example, the particular indication being treated, whether the desired benefit is prophylactic or therapeutic, the severity of the indication being treated and the age and weight of the patient, and the like. Determination of an effective dosage is well within the capabilities of those skilled in the art.

Effective dosages can be estimated initially from in vitro assays. For example, an initial dosage for use in animals can be formulated to achieve a circulating blood or serum concentration of active compound that is at or above an IC50 of the particular compound as measured in as in vitro assay, such as the in vitro fungal MIC or MFC and other in vitro assays described in the Examples section. Calculating dosages to achieve such circulating blood or serum concentrations taking into account the bioavailability of the particular compound is well within the capabilities of skilled artisans. For guidance, see Fingl & Woodbury, “General Principles,” In: Goodman and Gilman's The Pharmaceutical Basis of Therapeutics, Chapter 1, pp. 1-46, latest edition, Pagamonon Press, and the references cited therein, which are incorporated herein by reference.

Initial dosages also can be estimated from in vivo data, such as animal models. Animal models useful for testing the efficacy of compounds to treat or prevent the various diseases described above are well-known in the art.

The unit dosages described herein may be used in any appropriate dosing regimen.

As used here, a “binder” is a chemical compound that promotes cohesiveness of a powder such that powder can be transformed into granules through granulation where the active ingredient and excipients are bound together by the binder which gives the granule strength. Examples of binders suitable for granulating oteseconazole include, sucrose, gelatin, starch, certain cellulose derivatives (such as hydroxypropyl cellulose) and polyvinylpyrrolidone.

As used here, a “filler” is an inactive substance used to make the active medicine easier to measure. For example, fillers are often used in tablets or capsules because the amount of active drug is too small to be handled conveniently. Examples of fillers that may be suitable for granulating oteseconazole include lactose monohydrate and silicified microcrystalline cellulose.

As used here, a “disintegrant” is an excipient that is incorporated into the formulation of tablets or capsules to promote their disintegration when the tablet or capsule comes into contact with liquid or fluid matter. Examples of disintegrants that may be useful with the formulation of oteseconazole include starch, pregelatinized starch, croscarmellose sodium, crospovidone, sodium starch glycolate, alginic acid, calcium carboxymethylcellulose, polacrilin potassium, sodium starch glycolate, and the like.

As used here, the term “surfactant” refers to any compound capable (when used at an appropriate concentration) of decreasing the surface tension of an aqueous solution to a value of less than 50 mN/m, at room temperature. Surfactants may be anionic (e.g., sodium lauryl sulfate), nonionic, or cationic (e.g., compounds with a quaternized (i.e., tetra-substituted) ammonium group (e.g. N,N,N-trimethylhexadecan-1-ammonium chloride)).

### Study Design

Oteseconazole has been studied to compare the efficacy of oral oteseconazole with that of fluconazole in the treatment of acute VVC episodes in RVVC subjects. The dosing regimen described herein incorporate a loading dose phase to treat the initial acute infection with either fluconazole or oteseconazole. Fluconazole dosing regimen to be employed in the Loading dose Phase is in alignment with the recommendation proposed by the Infectious Disease Society of America (IDSA) for the treatment of vulvovaginal candidiasis, in which fluconazole, 150 mg, is given every 72 hours for a total of 3 doses. This recommendation is strongly supported by IDSA and for which high-quality evidence is available to support its clinical effectiveness.

An oral loading dose of about 600 mg of oteseconazole on Day 1 and about 450 mg of oteseconazole on Day 2 will provide for clinically effective plasma levels (approximately 2 μg/mL) to treat the presenting acute Candida spp. Infection and was shown to have efficacy comparable to that of fluconazole in the treatment of an acute VVC episode in subjects with RVVC. This dosing regimen and targeted oteseconazole plasma concentrations are expected to effectively treat Candida spp. that are generally resistant to fluconazole, including but not limited to C. glabrata, C. parapsilosis. C. krusei, C. tropicalis and fluconazole resistant C. albicans.

Oteseconazole dosing regimens will also incorporate a 150 mg once weekly maintenance dose phase for 11 weeks. The initial loading dose given on Days 1 and 2 combined with maintenance dosing of 150 mg oteseconazole weekly for a total treatment duration of 12 weeks was efficacious in preventing recurrence of acute VVC episodes for the duration of the study (48-50 weeks). Based on the PK results from a Phase 2b study and three Phase 3 studies, mean plasma concentrations of approximately 2.5 to 3.5 μg/mL are observed at the end of the dosing period (Week 14).

Oteseconazole has previously been evaluated in a Phase 2a multi-center, randomized, double-blind, active-controlled, parallel-group, dose-ranging study in patients with moderate-to-severe acute VVC. A total of 55 subjects with moderate-to-severe acute VVC (severity score ≥6 and a positive fungal KOH test) participated in the study across 4 dose groups: 1) oteseconazole 300 mg qd for 3 days: 2) 600 mg qd for 3 days: 3) 600 mg bid for 3 days; or 4) a single dose of fluconazole 150 mg followed by matching placebo. Oteseconazole was shown to be safe and well tolerated when administered for 3 days. There were no serious adverse events (AEs) reported and no treatment emergent adverse events (TEAEs) led to study discontinuation. No safety signals of clinical concern were observed from the safety assessments.

The efficacy of oteseconazole in the treatment of moderate-to-severe acute VVC was evaluated at the TOC visit in the Per Protocol population at the Day 28 test of cure (TOC) visit was, 87%, 86%, 86%, and 75% of subjects receiving the low, mid-, or high-dose oteseconazole or comparator arm of fluconazole, respectively, achieved an effective therapeutic cure (defined as having a total clinical signs and symptoms severity score of ≤1 and a negative culture for Candida species) in the Per Protocol population

### Formulations

All the excipients used in the formulations of oteseconazole are US, Japanese and European Pharmacopoeia grade. Water used in the granulation process is US, Japanese and European Pharmacopoeia grade. Oteseconazole was prepared in-house.

The anhydrous polymorph of oteseconazole (melting point about 103° C.) was used in the formulations disclosed in the present application. Details of the properties of the anhydrous form of oteseconazole is described in U.S. Pat. No. 10,414,751, the entirety of which is incorporated herein by reference.

The anhydrous polymorph of oteseconazole was micronized to control particle size of the drug substance. In one aspect, oteseconazole may be formulated as a tablet or a capsule. In another aspect, oteseconazole formulations comprise one or more excipients such as binders, fillers (e.g., calcium phosphate, microcrystalline cellulose, lactose, etc.), disintegrants and surfactants. The formulation may also include extra-granular excipients such as disintegrants and lubricant. Examples of lubricants suitable for formulating oteseconazole tablets and capsules include magnesium stearate, sodium lauryl sulfate and talc.

The following examples of pharmaceutical compositions comprising oteseconazole and a pharmaceutically acceptable carriers provide guidelines for how this active substance should be formulated. The skilled artisan would understand that deviations from the examples may still lead to pharmaceutically effective formulation. For example, the amount of the active and any ingredient/excipient present in any formulation described herein could independently be varied by as much as 50% provided that the formulation maintains its stability and effectiveness. For example, the amount of the active and any inactive ingredient (i.e., binder, filler, disintegrants, surfactant and lubricant) present in any formulation described herein may independently be varied by about 5%, about 10%, about 15%, about 20%, about 25%, about 30%, about 35%, about 40%, about 45%, or about 50%. Unless otherwise specified, the percentages refer to percentages by weight.

As used here, the term “about” means±10%. Thus, “about 25%” variation of an ingredient in any formulation means variation between 22.5% and 27.5% of the ingredient. Similarly, “about 40%” variation of an ingredient in any formulation means variation between 36% and 44% of the ingredient.

As used in connection with wt % of components in a particular formulation, the term “about” also means±10% of the amount of the ingredient in the formulation. For example, if an ingredient is stated to be present in a formulation at about 1 mg, such an ingredient is present in the formulation at between 0.9 and 1.1 mg. Similarly, if an ingredient is stated to be present in a formulation at about 5%, such an ingredient is present in the formulation at 5%±10% of 5% or between 4.5% and 5.5%.

A wet granulation manufacturing process was used to prepare the formulations. The process was designed to improve flowability and increase bulk density of the drug substance such that the size of the dosage form can be reduced to help with the ease of swallowing while providing good bioavailability. The drug substance was formulated as a tablet or a hard gelatin capsule, which is easy to swallow. The oteseconazole formulation described herein provides a small form tablet or capsule suitable for obtaining stable product using a production-scale manufacturing process. Table 1 provides a capsule composition comprising 150 mg oteseconazole.

TABLE 1
Composition of oteseconazole capsule, 150 mg

| Ingredient/Quality Standard | Function | Weight % | mg/capsule |
|---|---|---|---|
| Oteseconazole | Active | 75.0 | 150 |
| Silicified Microcrystalline Cellulose, NF, | Filler | 10.0 | 20 |
| Ph. Eur., JP | | | |
| Lactose Monohydrate, NF, Ph. Eur., JP | Filler | 6.0 | 12 |
| Hydroxypropyl Cellulose, NF, Ph. Eur., JP | Binder | 3.0 | 6 |
| Croscarmellose Sodium, NF, Ph. Eur., JP | Disintegrant | 2.0 | 4 |
| Sodium Lauryl Sulfate, NF, Ph. Eur., JP | Surfactant | 1.0 | 2 |
| Purified Water, USP^{b}, Ph. Eur., JP | Granulating | As required | As required |
| | Fluid | | |
| Total Intra-granular Weight | — | 97.0 | 194 |

Extra-granular Excipients

| | | | |
|---|---|---|---|
| Croscarmellose Sodium, NF, Ph. Eur., JP | Disintegrant | 2.0 | 4 |
| Magnesium Stearate, NF, Ph. Eur., JP | Lubricant | 1.0 | 2 |
| Total Blend Weight | — | 100.0 | 200 |

Capsule Shell

| Lavender size 2 capsule, OTE 150 printed | Capsule | 1 each |
|---|---|---|
| with black ink (Opacode SW-9008/SW- | Shell | |
| 9009) | | |

### Formulation Development

The first step in developing formulations was to determine whether excipients were compatible with oteseconazole for the purpose of developing a solid, immediate release oral dosage form of oteseconazole. Inventors identified that lactose (soluble filler), silicified microcrystalline cellulose (insoluble filler), croscarmellose sodium (disintegrant), hydroxypropyl cellulose (binder), sodium lauryl sulfate (wetting agent) and magnesium stearate (lubricant) were all suitable for preparing a solid, immediate release oral dosage form of oteseconazole. This formulation, fully described in Table 2, provides the framework for all subsequent formulations.

TABLE 2
Composition of oteseconazole clinical formulations and micronized oteseconazole
tablets and capsules, 150 mg and 300 mg.

| | | | | | | | | | | |
|---|---|---|---|---|---|---|---|---|---|---|
| | Ph 1 | Ph 1 | Phase 1 | Ph 2a | Ph 2a | Ph 2b | Ph 1 | Ph 1 | Ph 1/Ph 3 | |
| | Capsule | Capsule | Capsule | Tablet* | Tablet* | Tablet | Tablet | Capsule | Capsule | Tablet |
| Component | (mg) | (mg) | (mg) | (mg) | (mg) | (mg) | (mg) | (mg) | (mg) | (mg) |

Intra-granular Ingredients

| | | | | | | | | | | |
|---|---|---|---|---|---|---|---|---|---|---|
| Oteseconazole | 2.5^{a} | 20^{a} | 60^{a} | 50^{a} | 150^{a} | 150^{b} | 300^{b} | 150^{b} | 150^{b} | 150^{b} |
| Silicified Microcrystalline | 73.58 | 64.83 | 149.65 | 35.00 | 105.00 | 105.00 | 40 | 20 | 20 | 20 |
| Cellulose | | | | | | | | | | |
| Lactose Monohydrate | 74.37 | 65.62 | 151.45 | 74.25 | 222.75 | 222.75 | 24 | 12 | 12 | 12 |
| Hydroxypropyl Cellulose | 6.80 | 6.80 | 16.32 | 5.25 | 15.75 | 15.75 | 12 | 6 | 6 | 6 |
| Croscarmellose Sodium | 5.10 | 5.10 | 12.24 | 3.50 | 10.50 | 10.50 | 8 | 4 | 4 | 4 |
| Sodium Lauryl Sulfate | 1.70 | 1.70 | 4.08 | 1.75 | 5.25 | 5.25 | 4 | 2 | 2 | 2 |

Extra-granular Ingredients

| | | | | | | | | | | |
|---|---|---|---|---|---|---|---|---|---|---|
| Croscarmellose Sodium | 5.10 | 5.10 | 12.24 | 3.50 | 10.50 | 10.5 | 8 | 4 | 4 | 4 |
| Magnesium Stearate | 0.85 | 0.85 | 2.04 | 1.75 | 5.25 | 5.25 | 4 | 2 | 2 | 2 |
| Total Weight^{a} | 170 | 170 | 408 | 175 | 525 | 525 | 400 | 200 | 200 | 200 |

*Over-encapsulated into a size DB-A Swedish Orange capsule shell.
^{a}x-hydrate polymorphic form, un-micronized;
^{b}Form 1 (anhydrous), micronized

### Tablet Formulations

Tablets and capsules using anhydrous oteseconazole polymorph containing 150 mg (as described in Tables 1 and 2) meet the requisite high purity for a pharmaceutical drug product, are uniform and exhibit the expected dissolution profile.

Table 3 summarizes composition of 300 mg tablet formulations of oteseconazole. Table 4 provides bioavailability of the formulations described in Table 3. The composition of the 150 mg tablet and results of its relative bioavailability studies is included for reference. The first 300 mg formulation (Batch A) was prepared by doubling the drug load of the 150 mg strength tablet from 28.6% to 57.1% w/w. This was accomplished through a reduction in the quantity of the fillers (silicified microcrystalline cellulose and lactose), while keeping the total weight of the tablet unchanged at 525 mg. The quantity of the other excipients was not changed.

The second 300 mg formulation (Batch B) was similar to the first, the only difference being an increase in the level of extra-granular disintegrant from 2% to 3.9%. This resulted in a slight increase in the total tablet weight (535 mg versus 525 mg), but the amount of the other excipients in the formulation remained the same.

As described above, the drug load was double that of the 150 mg tablet for development Batches A and B. A single granulation was prepared for these two development batches using a laboratory scale, high shear granulator. The batch size was 125 g and 31% water relative to the dry solids was used for granulation. Drying was done in a fluid bed dryer using an inlet temperature of 60° C. and taken to a loss on drying (LOD) of 0.73%. The dried granules were split into two batches. In Batch A the extra-granular disintegrant was held at 2% and for Batch B it was increased to 3.9%. The batches were compressed manually using a Carver press.

Table 4 shows that the dissolution performance of both small-scale development batches was acceptable at all hardness levels evaluated. Minor differences in percent dissolved were observed at the 15-minute time point, with slightly slower dissolution for harder tablets and slightly faster initial dissolution for tablets with the higher disintegrant level. At the 30-minute time point, the release rates for all batches were comparable and the release nearly complete. Tablet disintegration times were slightly faster for tablets with lower hardness and/or with inclusion of additional disintegrant. The inventors contemplate that the extra-granular disintegrant level at 2% is sufficient for further development batches because the higher level of disintegrant did not appear to provide a significant benefit.

TABLE 3
Oteseconazole Tablets comprising 150 mg with decreased size of dosage form

| 150 mg Tablet | | 300 mg Tablet | | 300 mg Tablet | | 300 mg Tablet | | 300 mg Tablet | |
|---|---|---|---|---|---|---|---|---|---|
| Batch H | | Batch A | | Batch B | | Batch C | | Batches D, E and F | |
| mg/tab | wt % | mg/tab | wt % | mg/tab | wt % | mg/tab | wt % | mg/tab | wt % |

Intra-granular Excipients

| | | | | | | | | | | |
|---|---|---|---|---|---|---|---|---|---|---|
| Oteseconazole, | 150.00 | 28.6 | 300.00 | 57.1 | 300.00 | 56.0 | 300.00 | 66.7 | 300.00 | 75.0 |
| micronized | | | | | | | | | | |
| Silicified | 105.00 | 20.0 | 72.76 | 13.9 | 72.76 | 13.6 | 45.50 | 10.1 | 40.00 | 10.0 |
| Microcrystalline | | | | | | | | | | |
| Cellulose | | | | | | | | | | |
| Lactose | 222.75 | 42.4 | 105.00 | 20.0 | 105.00 | 19.6 | 64.00 | 14.2 | 24.00 | 6.0 |
| Monohydrate | | | | | | | | | | |
| Hydroxypropyl | 15.75 | 3.0 | 15.75 | 3.0 | 15.75 | 2.9 | 13.50 | 3.0 | 12.00 | 3.0 |
| Cellulose | | | | | | | | | | |
| Croscarmellose | 10.50 | 2.0 | 10.50 | 2.0 | 10.50 | 2.0 | 9.00 | 2.0 | 8.00 | 2.0 |
| Sodium | | | | | | | | | | |
| Sodium Lauryl | 5.25 | 1.0 | 5.25 | 1.0 | 5.25 | 1.0 | 4.50 | 1.0 | 4.00 | 1.0 |
| Sulfate | | | | | | | | | | |

Extra-granular Excipients

| | | | | | | | | | | |
|---|---|---|---|---|---|---|---|---|---|---|
| Croscarmellose | 10.50 | 2.0 | 10.50 | 2.0 | 21.00 | 3.9 | 9.00 | 2.0 | 8.00 | 2.0 |
| Sodium | | | | | | | | | | |
| Magnesium Stearate | 5.25 | 1.0 | 5.25 | 1.0 | 5.25 | 1.0 | 4.50 | 1.0 | 4.00 | 1.0 |
| Total Tablet Cores | 525.00 | 100.0 | 525.00 | 100.0 | 535.5 | 100.0 | 450.00 | 100.0 | 400.00 | 100.0 |

TABLE 4
Dissolution and Disintegration Data for Oteseconazole
Tablet, 300 mg, Development Batches A and B

| | Dissolution* | | | | |
|---|---|---|---|---|---|
| | (% Label Claim) | | | | Disintegration |
| | Avg 15 | Avg 30 | Avg 45 | Avg 60 | (time) |
| | min | min | min | min | Avg |
| Batch/hardness | (n = 3) | (n = 3) | (n = 3) | (n = 3) | (n = 3) |
| Batch-A, low | 78 | 97 | 96 | 97 | 10 min, 15 sec |
| hardness | | | | | |
| Batch-A, | 75 | 96 | 97 | 97 | 10 min, 25 sec |
| medium | | | | | |
| hardness | | | | | |
| Batch-A, high | 72 | 96 | 98 | 98 | 11 min, 45 sec |
| hardness | | | | | |
| Batch-B, low | 83 | 92 | 92 | 93 | 8 min, 36 sec |
| hardness | | | | | |
| Batch-B, | 85 | 94 | 94 | 94 | 9 min, 0 sec |
| medium | | | | | |
| hardness | | | | | |
| Batch-B, high | 72 | 92 | 93 | 93 | 10 min, 30 sec |
| hardness | | | | | |

*Dissolution conditions: 900 mL water with 2% SLS, 37° C., paddles at 75 rpm

The third 300 mg tablet formulation, shown in Table 3 as Batch C, was prepared by increasing the drug load from 57.1% to 66.7%. This was achieved by a further reduction in the quantity of the fillers resulting in a total tablet weight of 450 mg versus 525 mg. The tablets were made under the same granulation processing conditions as used for the first two development batches at UPM. Dissolution performance was acceptable for all hardness levels with complete release obtained by the 30-minute time point, as shown in Table 5. The disintegration times were slightly longer as compared to the lower drug load batches, but not significantly. Given this positive result, an additional increase the drug load of the tablet was evaluated.

TABLE 5
Dissolution and Disintegration Data for Oteseconazole
Tablet, 300 mg, Development Batch C

| | Dissolution* | | | | |
|---|---|---|---|---|---|
| | (% Label Claim) | | | | Disintegration |
| | Avg 15 | Avg 30 | Avg 45 | Avg 60 | (time) |
| | min | min | min | min | Avg |
| Batch | (n = 2) | (n = 2) | (n = 2) | (n = 2) | (n = 3) |
| Batch C, low | 69 | 99 | 101 | 102 | 11 min, 27 sec |
| hardness | | | | | |
| Batch C, | 80 | 101 | 102 | 102 | 11 min, 50 sec |
| medium | | | | | |
| hardness | | | | | |
| Batch C, high | 68 | 99 | 101 | 102 | 12 min, 28 sec |
| hardness | | | | | |

*Dissolution conditions: 900 mL water with 2% SLS, 37° C., paddles at 75 rpm

The fourth and final 300 mg formulation (Batches D, E, and F, see Table 3) was prepared by increasing the drug load to 75% through a reduction in the quantity of the fillers to further reduce the total tablet weight to 400 mg. The level of the binder, disintegrant, wetting agent and lubricant were kept constant relative to the total tablet weight.

This formulation was first evaluated in a small screening study for Batch numbers D and E. The same input granulation was used for both batches. Batch D was compressed on the manual Carver tablet press and Batch E was compressed on the automated Styl'One tablet press. Dissolution performance for tablets compressed on either press were acceptable and comparable. The disintegration times were slightly longer as compared to a lower drug loaded batch, but this was not considered significant. On the Styl'One use of compression forces as high as 24 kN did not result in harder tablets. Tablet hardness reached a plateau in the range of 10-15 KP, so only tablets with two different hardness levels were assessed. Based on the promise of this 75% drug loaded formulation, it was scaled up to an 800 g batch size for further evaluation.

A small scale demonstration batch of the 75% drug loaded tablet formulation was manufactured. Table 5 shows the assay values, content uniformity and dissolution results for Oteseconazole Tablet, 300 mg, Batch F (800 g batch size) compressed using a force of 2.6 KN. The average hardness was 11.1 kp (range 10.2-12.3 kp) and friability was 0.1% w/w. Assay was 100.7%, content uniformity had a mean value of 100.9% with a range from 99.5% to 102.4% and dissolution was complete within 60 minutes.

TABLE 6
Assay, Content Uniformity and Dissolution Data
for Oteseconazole Tablet, 300 mg, Batch F

| Assay | | Content Uniformity | | Dissolution at 60 minutes* | |
|---|---|---|---|---|---|
| (% LC) | | (% LC) | | (% LC) | |
| Sample 1 | 100.8 | Sample 1 | 100.5 | Vessel 1 | 97 |
| Sample 2 | 100.7 | Sample 2 | 100.8 | Vessel 2 | 99 |
| Sample 3 | 100.5 | Sample 3 | 102.4 | Vessel 3 | 99 |
| Sample 4 | 100.1 | Sample 4 | 99.5 | Vessel 4 | 96 |
| Sample 5 | 101.0 | Sample 5 | 101.3 | Vessel 5 | 97 |
| Sample 6 | 100.9 | Sample 6 | 100.4 | Vessel 6 | 97 |
| Mean | 100.7 | Sample 7 | 100.7 | Mean | 97 |
| RSD | 0.3% | Sample 8 | 102.3 | RSD | 1.1 |
| | | Sample 9 | 100.9 | | |
| | | Sample 10 | 100.4 | | |
| | | Mean | 100.9 | | |
| | | RSD | 0.9 | | |
| | | AV | 2.1 | | |

*Dissolution conditions: 900 mL water with 2% SLS, 37° C., paddles at 75 rpm

### Capsule Formulations

Oteseconazole capsules 150 mg strength using the 75% drug loaded formulation developed for oteseconazole tablets were prepared, decreasing the target capsule fill weight from 400 mg to 200 mg. The tablet formulation was deemed suitable for use in a capsule, since tablet hardness did not have a significant impact on dissolution profile. Oteseconazole Capsule, 150 mg, Batch G was successfully manufactured using the 75% drug loaded formulation described in Table 3.

### Summary of Relative Bioavailability Study Oteseconazole CL-013

The 150 mg strength capsule formulation used in the relative bioavailability study is fully described in Table 2 for bulk capsule Batch G. No changes in this formulation were made for the Phase 3 capsule Batch (bulk Batch I).

Relative bioavailability study Oteseconazole CL-013 compared the bioavailability of the final 150 mg capsule formulation (bulk capsule Batch G) to the Phase 2b tablet formulation (Batch H) under fed (high fat) conditions. In addition, the capsule formulation was also administered in the fasted state and with a low-fat meal. The summary of the pharmacokinetic results from this study are shown in Table 7. There was no significant difference in bioavailability between the capsule and tablet when given with a high fat meal. In addition, there was no clinically significant food effect for the capsule, suggesting that oteseconazole is highly absorbed from the capsule formulation.

TABLE 7
Pharmacokinetic Summary for Study Oteseconazole CL-013

| | | | | LSM | LSM |
|---|---|---|---|---|---|
| | Tmax | Cmax | AUC0-72 | Ratio | Ratio |
| Treatment | (h) | (ng/mL) | (ng · h/mL) | Cmax | AUC0-72 |
| Capsule* (fasted) | 5.0 | 563 | 17,580 | Ref | Ref |
| Capsule* (low fat) | 4.9 | 637 | 17,590 | 1.17 | 1.02 |
| Capsule* (high fat) | 6.7 | 821 | 22,970 | 1.45 | 1.29 |
| Tablet† (high fat) | 5.3 | 805 | 21,310 | n.d. | n.d. |

*Batch G;
†Batch H

### Batch Formula

The batch formula for Oteseconazole Capsule, 150 mg is provided in Table 8. The proposed commercial batch size is 85 kg, representing 425,000 capsules.

TABLE 8
Oteseconazole Capsule, 150 mg Batch Formula

| | | Percent | Batch |
|---|---|---|---|
| | | Composition | Quantity |
| Ingredient/Quality Standard | mg/capsule | (% w/w) | (kg) |
| Oteseconazole^{a}, Internal | 150 | 75.00 | 63.75 |
| Silicified Microcrystalline Cellulose^{a}, NF, | 20 | 10.00 | 8.500 |
| Ph. Eur., JP | | | |
| Lactose Monohydrate, NF, Ph. Eur., JP | 12 | 6.00 | 5.100 |
| Hydroxypropyl Cellulose, NF, Ph. Eur., JP | 6 | 3.00 | 2.550 |
| Croscarmellose Sodium, NF, Ph. Eur., JP | 4 | 2.00 | 1.700 |
| Sodium Lauryl Sulfate, NF, Ph. Eur., JP | 2 | 1.00 | 0.850 |
| Purified Water, USP^{b}, Ph. Eur., JP | As required ^{b} | N/A ^{b} | 22.95 ^{b} |
| Total Intra-granular | 194 | 97.00 | 82.45 |

Extra-granular Excipients

| | | | |
|---|---|---|---|
| Croscarmellose Sodium, NF, Ph. Eur., JP | 4 | 2.00 | 1.700 |
| Magnesium Stearate, NF, Ph. Eur., JP | 2 | 1.00 | 0.850 |
| Total Blend Weight | 200 | 100.00 | 85.00 |

Capsule Shell

| | | | |
|---|---|---|---|
| Lavender size 2 capsule^{c}, printing with | 1 each | N/A | 425,000 |
| black ink (Opacode SW-9008/SW-9009) | | | capsules |

^{a}The amount of drug substance is adjusted based on the weight percentage of active ingredient in the particular lot. The corresponding amount of silicified microcrystalline cellulose is adjusted to maintain the desired capsule weight.
^{b} Removed during processing.
^{c}The lavender opaque gelatin capsule shell consists of FD&C Blue #1, FD&C Red #3, Titanium Dioxide and Gelatin (qsp 100%).
N/A = Not applicable, removed during drying.

### Physicochemical and Biological Properties

Oteseconazole is a BCS Class IIc drug (low solubility, high permeability, unionized throughout the pH range of the gastrointestinal tract). As a BCS Class IIc drug, it may be anticipated that dissolution may be dependent upon the drug substance particle size. (Tsume et al., 2014). Micronization of the drug substance provided a means to deliver drug substance with a small and consistent particle size. A discriminatory dissolution method was developed to show the effect of drug substance particle size on dissolution to help ensure product quality. Micronization of oteseconazole drug substance resulted in it having a low bulk density, approximately 0.2 g/mL. A high shear wet granulation process was developed to densify the product, and improve its flow to enable manufacturing, while maintaining good dissolution from the formulation. Oteseconazole is slightly hygroscopic, stable in the presence of light and has excellent chemical stability both alone and when formulated into a drug product.

### Description of Manufacturing Process and Process Controls

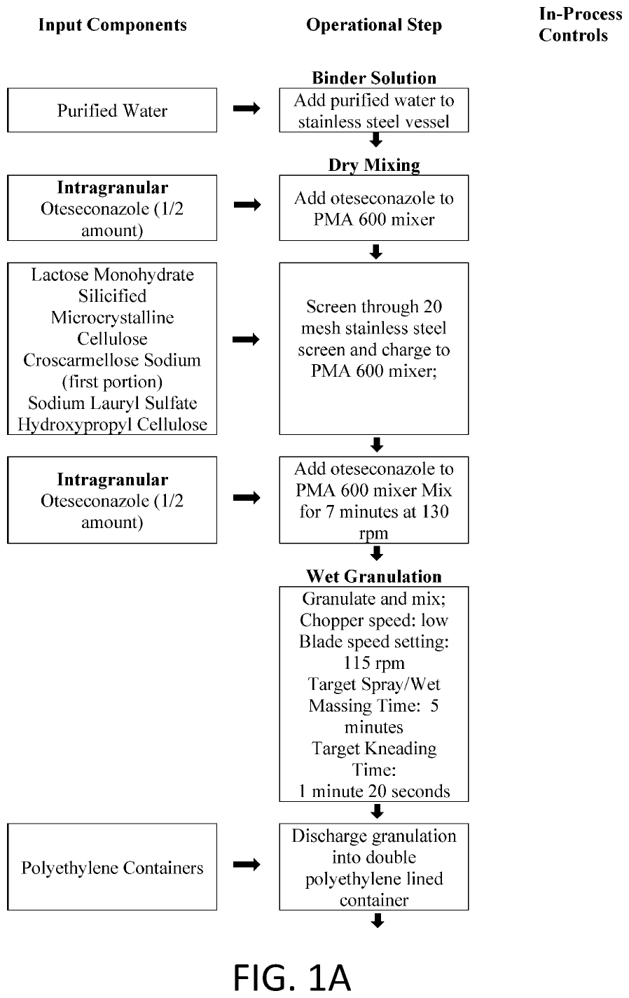

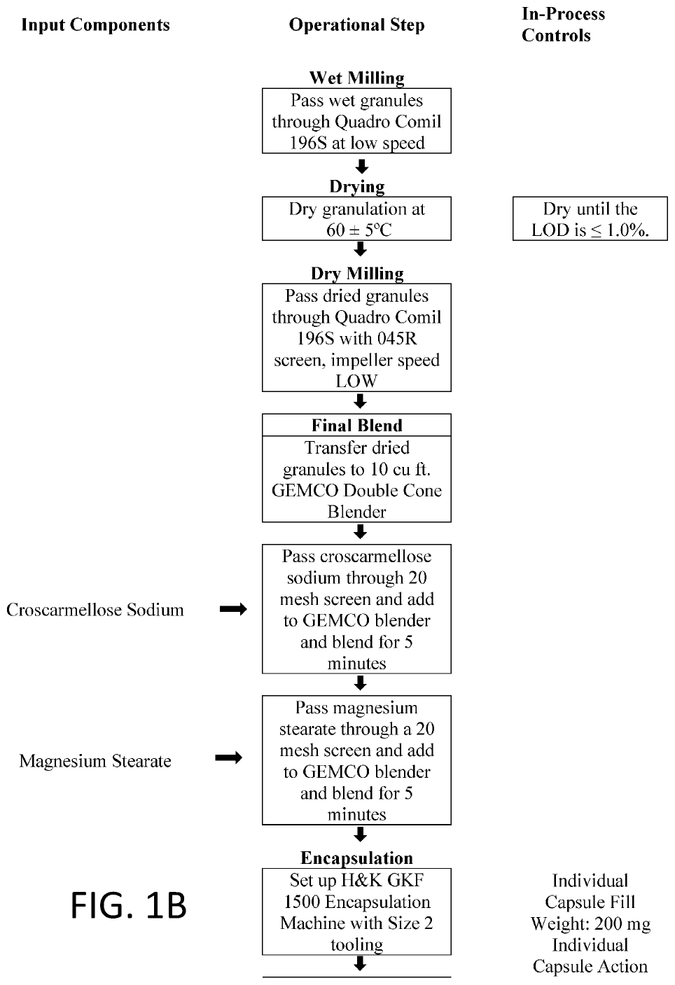

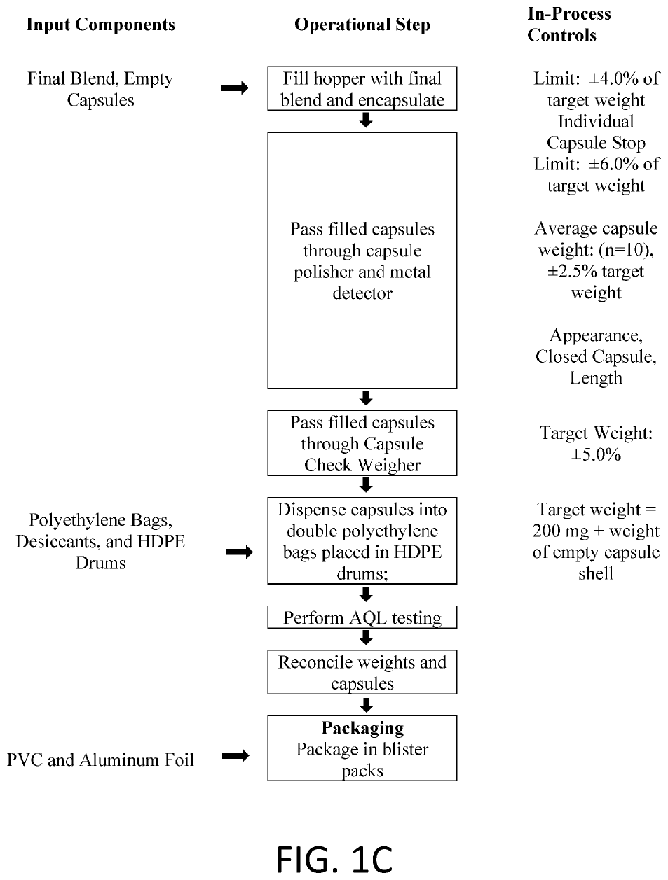

The proposed commercial manufacturing process for manufacture of Oteseconazole Capsule, 150 mg is provided in FIG. 1, followed by a narrative of the process.

### Manufacturing

Although the manufacturing procedure described below uses a present tense, the procedure was in fact completed and its utility confirmed an inventor of the present application or by someone working under the guidance of an inventor.

### Binder Solution

1. Prepare the binder solution by charging the required quantity of purified water into a suitable stainless steel vessel. Calibrate the spray system to deliver purified water at a specified rate of approximately 4.6 kg/minute.

### Weighing of the Components

2. The excipients and the drug substance are individually weighed and charged into a PMA-600 high shear mixer granulator.

### Dry Mixing

3. Oteseconazole is dry mixed with the silicified microcrystalline cellulose, lactose monohydrate, sodium croscarmellose, hydroxypropyl cellulose and sodium lauryl sulfate according to the following target settings:
   a. Main impeller speed: 130 rpm
   b. Chopper: Off
   c. Mixing time: 7 minutes

### Wet Granulation

4. Granulate using purified water (22.95 kg) as indicated below:
   a. Chopper speed setting: low
   b. Main impeller speed: 115 rpm
   c. Target Spray Time: 5 minutes
5. Open the PMA600 mixer and manually scrape down any material adhering to the granulator walls.
6. Continue to mix as indicated below:
   a. Chopper speed setting: low
   b. Main impeller: 115 rpm
   c. Mixing Time: 1 minute, 20 seconds
7. Open the PMA600 mixer and manually scrape down any material adhering to the granulator walls.
8. Discharge granulation from the PMA600 into double polyethylene lined containers. During discharge, stop the mixer, open the bowl, and scrape the bowl to facilitate transfer.

### Wet Milling

9. Pass the wet granules through a Quadro Comil 196S (250Q Screen, Round Impeller) at LOW speed into an appropriate container.

### Drying

10. Dry the granulation in the Glatt Fluid Bed Dryer, model WST-120 using an inlet temperature of 60±5° C. Dry until the LOD is ≤1.0%. This In-Process Control target is typically reached when a product temperature of 50° C. is reached when using an Inlet Air temperature of 60° C.

### Dry Milling

11. Pass the dried granulation through a Quadro Comil 196S with a 045R screen, round impeller at LOW speed and into a suitable container

### Final Blending and Lubrication

12. Reconcile the weights and adjust the amount of the extra-granular excipients (Croscarmellose Sodium and Magnesium Stearate) as needed.
13. Transfer the dried granules to a 10 cu. Ft. GEMCO Double Cone Blender.
14. Pass the croscarmellose sodium needed for the final blend through a 20 mesh screen and add to the GEMCO Double Cone Blender.
15. Blend the croscarmellose sodium with the dried granulation for 5 minutes.
16. Pass the magnesium stearate needed for the final blend through a 20 mesh screen and add to the GEMCO Double Cone Blender.
17. Blend the magnesium stearate with the dried granulation for 5 minutes and discharge into a suitable container.

### Encapsulation

18. Set up the H&K GKF 1500 Encapsulating Machine with size 2 tooling and a suitable mm dosing disk.
19. Fill the hopper with the final blend and encapsulate with the following targets:
   a. Individual capsule fill weight: 200 mg
   b. Individual capsule weight, ±6.0% of target weight*
   c. Average capsule weight (n=10), ±2.5% of target weight*
20. After adjustments to achieve these weight targets, perform a visual inspection of initial capsules.
21. Encapsulate the remaining blend.
22. Pass the filled capsules through the capsule polisher equipped with an empty capsule eliminator.
23. Pass the polished capsules through a metal detector.
24. Pass the filled capsules through a Capsule Check Weigher set at a target weight* of ±5% as calculated on the Weight Specifications Calculation page.
25. Periodically perform in-process tests on weight, capsule length and appearance.
26. Capsules are loaded into the bulk drums.
27. Perform AQL inspection on weight sorted capsules.
28. Reconcile the weights and capsules.
   *Target weight=200 mg+weight of empty capsule shell
   Note: The empty capsule shell weight is approximately 61 mg, but its average weight is determined prior to encapsulation as part of the batch manufacturing process.

### Bulk Packaging

The finished capsules are bulk-packaged into double polyethylene bags. The bags are sealed with zip ties and placed into suitable HDPE containers. The containers are then placed in quarantine and moved to GMP storage pending disposition for final packaging into blister packs.

The bulk capsules may be held for up to 9 months.

### Packaging

The finished capsules are packed in blister packs that will be inside a child-resistant cardboard wallet, which is placed within an outer cardboard carton. The blister pack is comprised of a clear film and a push-through lidding. The clear film is made using a thermoformable rigid polyvinyl chloride (PVC) film suitable for pharmaceutical packaging. The push-through blister lidding of the blister pack is composed of aluminum foil and lacquers typically used in pharmaceutical packaging

### Pharmacokinetics Studies

### Abbreviations:

AUC0−x=area under the plasma concentration-time curve from 0 to x hours;
max=maximum;
min=minimum;
PK=pharmacokinetic(s);
SD=standard deviation;
Tmax=time to reach maximum concentration (presented as median [N] (min. max));
AUC0−∞=area under the plasma concentration versus time curve from time 0 extrapolated to
infinity;
CL/F=apparent clearance;
Cmax=maximum measured plasma concentration;
PK=pharmacokinetics;
t½=apparent terminal half-life;
Tlag=lag time for absorption after dosing;
Vz/F=apparent volume of distribution
AUCτ=area under the plasma concentration versus time curve from time 0 to time (t);
Cavg=average concentration;
Cmin=minimum measure plasma concentration;
Racc, accumulation ratio;
CV=coefficient of variation;
DDI=drug-drug interaction;
PKE-pharmacokinetic evaluation;
EE-ethinyl estradiol;
MD=multiple-dose;
N=norethindrone;
SD=single-dose;
Clast=last observed quantifiable concentration;
QD=once daily;
QW=once weekly
NC=not calculated;
BMI=body mass index;
EU=European Union;
ISE=Integrated Summary of Efficacy;
US=United States.

### Single-Dose Escalation Studies in Healthy Subjects (Oteseconazole CL-001)

Study Oteseconazole CL-001 (hereafter referenced as “CL-001”) was a single-center, Phase 1, double-blind, randomized, placebo-controlled, dose-escalating study in healthy adult subjects.

Sixty-four subjects (43 males and 21 females) between 18 and 55 years of age were enrolled in 8 dose groups. Within each dose group, 6 subjects received oteseconazole and 2 subjects received placebo. In the first 7 cohorts, escalating single oral doses (5, 10, 20, 40, 80, 160, or 320 mg) were administered in the fasted state using 2.5, 20, or 60 mg capsules of oteseconazole or matching placebo. An eighth cohort received a single 320 mg oral dose of oteseconazole or matching placebo after ingesting a high-fat breakfast. Subjects in Cohorts 1 through 6 were followed for 14 days for safety and PK assessments. Visits on Days 28 and 42 were added for Cohorts 7 and 8. Dose escalation proceeded only after safety and PK of the previous dose were determined.

The PK analysis was based on concentration-time data from 36 subjects who received active oteseconazole at doses between 20 mg and 320 mg. The majority of the oteseconazole plasma concentrations in Cohort 1 (5 mg) and Cohort 2 (10 mg) were below the limit of quantification (<20 ng/mL); therefore, no PK analysis could be conducted on these subjects.

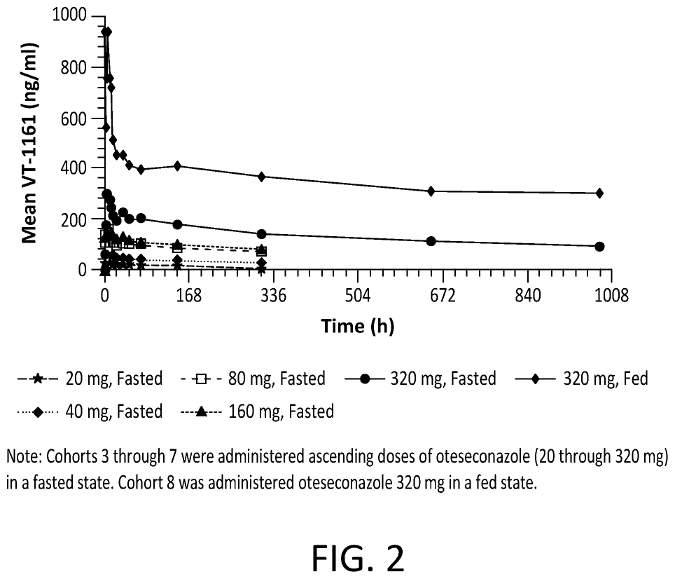

A summary of the PK results is presented in Table 9. The mean oteseconazole plasma concentration-time plots for Cohorts 3 through 8 are presented in FIG. 2.

Following oral administration of oteseconazole in the fasted state, most subjects displayed a lag time for absorption after dosing (T_{lag}) ranging from 0.5 hours to 2 hours. Following the maximum concentration (Cₘₐₓ), the concentrations decreased for several hours and were then relatively constant up to 984 hours postdose (in subjects with long sampling times), due to the long half-life of oteseconazole.

Median time to reach maximum concentration (Tₘₐₓ) values ranged from 4 hours (80 mg) to 10 hours (40 mg), indicating that oteseconazole is relatively slowly absorbed. The average Cₘₐₓ increased in proportion to the dose for doses ranging from 20 mg to 80 mg, was less than in proportion to the increase in dose from 80 mg to 160 mg, and was again in proportion to the dose as the dose was increased from 160 mg to 320 mg. Overall, the relationship between oteseconazole dose and Cₘₐₓ indicated that Cₘₐₓ increased slightly less than proportionally to the increase in dose.

The area under the plasma concentration versus time curve from time 0 extrapolated to infinity (AUC_{0-∞}) values generally increased with increase in dose, ranging from an average of 16145 h·ng/mL at 20 mg to 241971 h·ng/mL at 320 mg. Inter-subject variability was moderate at all dose levels, with coefficients of variation (CV) ranging from 15% to 43%. The relationship between oteseconazole dose and AUC_{0-∞} was generally linear with the exception for the dose escalation between 80 and 160 mg. The apparent clearance (CL/F) values were moderate and similar for all cohorts, with averages ranging from 1.08 to 1.67 L/h and appeared to be independent of administered dose. The apparent volume of distribution (V_{z}/F) values were very large, with average values ranging from 696 to 1649 L, and there was a trend for V_{z}/F to increase with dose.

Accurate estimates of t_{1/2} were limited by the number of data points in the terminal phase, although blood sampling was carried out for 312 hours for Cohorts 3 through 6 and 984 hours for Cohorts 7 and 8. Despite these long sampling times, robust estimates were not possible. The median half-lives were estimated at 393, 548, 536, 618, 873, and 1467 hours for Cohorts 3 through 8, respectively.

Administration of 320 mg oteseconazole with a standard high-fat meal (Cohort 8) resulted in higher plasma concentrations than those observed following 320 mg in the fasted state in a separate group of subjects (Cohort 7). The summary of the pharmacokinetic results from this study are shown in Table 9.

TABLE 9
Oteseconazole pk parameters (CL-001)

| | | Dose | Cₘₐₓ | Tₘₐₓ | T_{lag} | t_{1/2} | AUC_{0-∞} | CL/F | Vz/F |
|---|---|---|---|---|---|---|---|---|---|
| Cohort | N | (mg) | (ng/mL) | (h) | (h) | (h) | (h*ng/ml) | (L/h) | (L) |
| 3 | 6 | 20 | 44.7 | 5.03 | 1.84 | 393 ^{a} | 16145 ^{a} | 1.39 ^{a} | 696 ^{a} |
| | | | (29.5) | (4.00, | (32.9) | (42.2) | (39.3) | (35.2) | (17.7) |
| | | | | 6.00) | | | | | |
| 4 | 6 | 40 | 79.7 | 10.00 | 1.58 | 548 | 35037 | 1.30 | 939 |
| | | | (33.2) | (6.00, | (23.8) | (33.3) | (43.2) | (38.8) | (24.2) |
| | | | | 12.00) | | | | | |
| 5 | 6 | 80 | 165 | 4.01 | 1.17 | 536 | 84635 | 1.08 | 761 |
| | | | (31.0) | (4.00, | (44.1) | (41.0) | (41.6) | (37.8) | (23.8) |
| | | | | 6.00) | | | | | |
| 6 | 6 | 160 | 180 | 6.00 | 0.75 | 618 | 108869 | 1.67 | 1397 |
| | | | (27.3) | (2.00, | (36.5) | (27.4) | (34.0) | (43.6) | (29.7) |
| | | | | 10.02) | | | | | |
| 7 | 6 | 320 | 338 | 5.00 | 0.67 | 873 | 241971 | 1.35 | 1649 |
| | | Fasted | (29.8) | (4.00, | (38.7) | (29.9) | (14.5) | (18.2) | (21.2) |
| | | | | 10.00) | | | | | |
| 8 | 6 | 320 | 988 | 6.00 | 1.25 | 1467 | 953119 | 0.406 | 741 |
| | | Fed | (30.9) | (4.00, | (42.0) | (43.7) | (52.6) | (42.0) | (17.4) |
| | | | | 10.07) | | | | | |

^{a} N = 5.
Note:
Units are mean (CV %) for all parameters except Tₘₐₓ (median [minimum, maximum]).

Study in Subjects with Tinea Pedis (Oteseconazole CL-003)

Oteseconazole was slowly absorbed, with median time to peak plasma concentration values that ranged from 5.0 to 6.0 hours after dosing on Days 1 and 14 for all treatments. The Cₘₐₓ increased less than proportionally to dose for the low and mid-dose treatments on both Days 1 and 14, where a 3-fold increase in dose resulted in a 2-fold increase in concentration. The Cₘₐₓ values on Day 1 were similar between the mid- and high-dose treatments, since the first dose on that day was 600 mg for each treatment. Although there was a 2-fold dose difference between the mid- and high-dose treatments on Day 14, there was a modest, 15% increase in the Cₘₐₓ value. Based on the Cₘₐₓ values observed in the study, the target was met for the low dose group, but the mid-dose group only achieved about 50% of target and the high-dose group only about 25% of target (Table 10).

The AUC₀₋₈ values on Days 1 and 14 displayed similar relationships between dose groups, with less than proportional increases as the doses were increased. Results of the terminal phase half-life assessment based on blood sampling carried out for 168 days (4032 hours) confirmed that the half-life of oteseconazole is very long, with median values ranging from 2081 hours to 4128 hours.

TABLE 10
Oteseconazole Pharmacokinetic Parameters (CL-003)

| | Day 1 | | | Day 14 | | |
|---|---|---|---|---|---|---|
| | Cₘₐₓ | | AUC₀₋₈ | Cₘₐₓ | | AUC₀₋₈ |
| | (ng/ | Tₘₐₓ | (hr*ng/ | (ng/ | Tₘₐₓ | (hr*ng/ |
| Treatment | mL) | (hr) | ml) | mL) | (hr) | mL) |
| Low Dose | 366 | 6.00 | 1680 | 1090 | 5.00 | 7950 |
| 200 mg/50 mg | (33.9) | (3.00, | (33.9) | (47.6) | (4.00- | (50.0) |
| | | 8.00) | | | 8.02) | |
| Mid Dose | 679 | 6.00 | 3320 | 2180 | 5.00 | 16100 |
| 600 mg/150 mg | (40.3) | (5.00, | (42.2) | (39.7) | (2.00- | (39.8) |
| | | 8.00) | | | 8.00) | |
| High Dose | 757 | 5.00 | 3540 | 2520 | 5.02 | 17600 |
| 1200 mg/300 mg | (37.4) | (4.00, | (30.4) | (50.6) | (2.00- | (49.5) |
| | | 6.00) | | | 8.00) | |

Even though the target concentrations were not reached for either dose group in this study, oteseconazole was efficacious in treating TP. Results were variable, as therapeutic cure was achieved in 16.7%, 8.3%, and 21.4% in the low, mid, and high dose groups, respectively, versus 0% in the placebo group.

### Study Oteseconazole CL-004

Oteseconazole was slowly absorbed, with a median Tₘₐₓ of 4.0 hours for the 300 mg oteseconazole dose and 5.5 hours for the 600 mg oteseconazole dose. On Day 1, the Cₘₐₓ increased in an approximate linear fashion from 407 ng/ml for the 300 mg dose to 968 ng/ml for the 600 mg dose. Similarly, area under the plasma concentration versus time curve from 0 to 8 hours (AUC₀₋₈) values on Day 1 also increased approximately proportional to the increase in dose, averaging 1,870 and 4230 h*ng/mL for the 300 and 600 mg doses, respectively.

The ty, values were estimated on the basis of blood sampling that was carried out between approximately 7 and 168 days after administration of the first dose. The ty, were shown to be very long, with median t_{1/2} ranging from 1822 hours for the 300 mg QD treatment group, 3192 hours for the 600 mg QD treatment group, and 3840 hours for the 600 mg BID treatment group. The oteseconazole concentrations on Day 168 remained measurable (127, 409, and 1300 ng/mL for the 300 mg QD, 600 mg QD, and 600 mg BID treatment groups, respectively) and remained at or above therapeutic concentrations for the 600 mg QD and 600 mg BID treatment groups.

The predicted minimum concentration (Cₘᵢₙ) at 24 hours after the final Day 3 dose indicated that the Cₘᵢₙ increased with increasing dose, averaging 705, 1000, and 2830 ng/mL for the 300 mg QD, 600 mg QD, and 600 mg BID dose groups, respectively. Based on the calculated Cₘᵢₙ values, only about 50% of the target was met for the 300 mg QD and 600 mg QD treatment groups, with the 600 mg BID treatment group reaching the target anticipated for the 600 mg QD group.

Study in Subjects with Onychomycosis (Oteseconazole CL-005)

Oteseconazole plasma exposures increased approximately proportional to dose after 12 weeks in the 300 mg 12-week (6.75 μg/mL) and 600 mg 12-week (12.3 μg/mL) treatment groups and after 24 weeks in the 300 mg 24-week (9.23 μg/mL) and 600 mg 24-week (15.6 μg/mL) treatment groups. Within each treatment group, oteseconazole plasma exposures increased throughout the dosing regimen from Day 1 to Week 12 or 24. In all 4 treatment groups, oteseconazole plasma exposures slowly decreased from the time of the final dose of oteseconazole through the Week 60 Visit.

Mean Cₘₐₓ and AUC₀₋₈ values after the Day 14 dose (completion of the loading dose phase of the study) were similar for the 300 mg 12-week and 300 mg 24-week treatment groups, and for the 600 mg 12-week and 600 mg 24-week treatment groups, as expected. The calculated PK parameters for oteseconazole in plasma for each treatment group are presented in Table 11.

TABLE 11
Plasma Pharmacokinetic Parameters of Oteseconazole on Day 14 (CL-005)

| | Oteseconazole Dose, Treatment Duration | | | | | | | |
|---|---|---|---|---|---|---|---|---|
| | Low Dose (300 mg) | | | | High Dose (600 mg) | | | |
| PK Parameter | 12 weeks | | 24 weeks | | 12 weeks | | 24 weeks | |
| Cₘₐₓ (μg/mL) | 5.12 | (2.10) | 5.51 | (1.35) | 8.29 | (1.90) | 8.39 | (4.86) |
| (SD) | | | | | | | | |
| Tₘₐₓ (hr) | 5.00 | (4.00, 6.08) | 5.00 | (4.00, 5.00) | 5.00 | (4.00, 8.00) | 5.00 | (2.00, 8.00) |
| (min, max) | | | | | | | | |
| AUC₀₋₈ | 36.7 | (15.1) | 40.1 | (9.54) | 59.7 | (14.1) | 61.6 | (37.3) |
| (μg*hr/mL) | | | | | | | | |
| (SD) | | | | | | | | |

The 4 dosing regimens yielded oteseconazole plasma concentrations that resulted in positive efficacy outcomes across all dose groups, with no major differences in the extent of efficacy noted. For the 300 mg and 600 mg 12- and 24-week oteseconazole dose regimen, complete cure ranged between 32% and 42% with no corresponding dose response.

### Study Oteseconazole CL-006

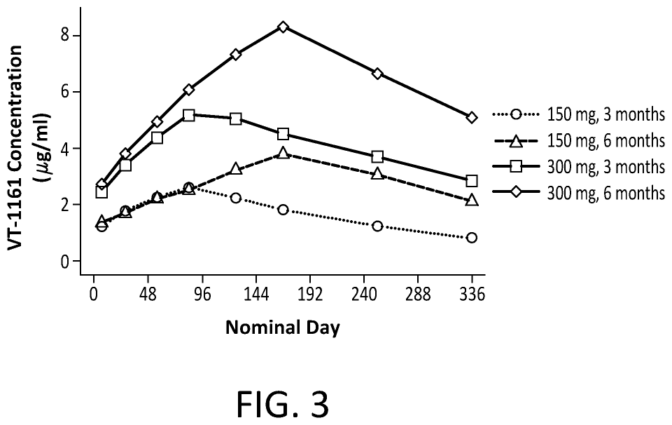

Oteseconazole plasma concentrations continued to increase after the 7-day loading dose portion of the Maintenance Period until the end of dosing in an approximately dose-proportional manner FIG. 3. After the end of dosing, oteseconazole concentrations slowly decreased to concentrations that were still well above the minimum concentration to inhibit growth of 90% of organisms (MIC₉₀) for C. albicans at Week 48 (2.15 μg/mL and 5.09 μg/mL for 150 mg and 300 mg oteseconazole, respectively). The half-life of oteseconazole was calculated using a population PK approach to be approximately 4100 hours.

Mean plasma PK parameters on Day 7 of the Maintenance Period are presented by treatment group in Table 12. In the serial PK analysis population, mean Cₘₐₓ and AUC₀₋₈ values after the Day 7 dose (completion of the Induction Phase of the study) were similar for the 150 mg 12 weeks and 150 mg 24 weeks treatment groups, and for the 300 mg 12 weeks and 300 mg 24 weeks treatment groups, as expected.

TABLE 12
Plasma Pharmacokinetic Parameters of Oteseconazole on Day 7 (CL-006)

| | Oteseconazole Dose, Treatment Duration | | | | | | | |
|---|---|---|---|---|---|---|---|---|
| | 150 mg; 12 | | 150 mg; 24 | | 300 mg; 12 | | 300 mg; 24 | |
| PK Parameter | weeks | | weeks | | weeks | | weeks | |
| Cₘₐₓ (μg/mL) | 1.34 | (1.02) | 1.80 | (1.06) | 2.75 | (1.13) | 3.12 | (1.28) |
| (SD) | | | | | | | | |
| Tₘₐₓ (hr) | 4.00 | (0.00, 6.00) | 4.93 | (2.00, 5.00) | 4.92 | (2.00, 6.00) | 5.00 | (2.00, 8.00) |
| (min, max) | | | | | | | | |
| AUC₀₋₈ | 9.12 | (7.25) | 12.3 | (6.30) | 20.5 | (6.02) | 21.6 | (9.14) |
| (μg*hr/mL) | | | | | | | | |
| (SD) | | | | | | | | |

The dosing regimen yielded oteseconazole plasma concentrations that provided marked efficacy across all dose groups, with no differences in the extent of efficacy noted. For the 150 mg, 3 months dose group, only 2 subjects (4.8%) had a culture-verified recurrent VVC episode by Week 48, versus 24 subjects (52.2%) reaching this endpoint in the placebo group. The loading dose of 150 mg oteseconazole QD for 7 days provided adequate oteseconazole concentrations to avoid early recurrences, and continued maintenance dosing at 150 mg oteseconazole QW for 11 weeks resulted in sustained efficacy.

### Relative Bioavailability Study (Oteseconazole CL-007)

Oteseconazole plasma exposure in the DDI cohort increased from Day 3 (geometric mean Cₘₐₓ of 1913 ng/ml) to Day 16 (geometric mean Cₘₐₓ of 11570 ng/ml). The median Tₘₐₓ was 4.0 hours on Day 3 and 6.0 hours on Day 16. The geometric mean AUC₀₋₂₄ increased from 24610 h*ng/mL on Day 3 to 243200 h*ng/mL on Day 16, and the geometric mean AUC_{0-∞} increased from 49390 h*ng/mL on Day 3 to 4881000 h*ng/mL on Day 16. The increase in oteseconazole concentrations after 14 days of daily dosing was expected based on the long half-life of oteseconazole and are a consequence of accumulation and not of concomitant administration of midazolam.

In the PKE cohort, the geometric mean Cₘₐₓ was 1903 ng/ml and the median Tₘₐₓ was 6.0 hours. The geometric mean AUC0-24 was 26120 h*ng/ml and the geometric mean AUC_{0-∞} was 57290 h*ng/mL. These values were consistent with the oteseconazole plasma PK parameters calculated on Day 3 in the DDI cohort.

TABLE 13
Oteseconazole Pharmacokinetic Parameters in DDI and PKE Cohorts (CL-007)

| | DDI Cohort | | | | PKE Cohort | |
|---|---|---|---|---|---|---|
| PK Parameter | Day 3 | | Day 16 | | Day 1 | |
| AUC₀₋₂₄ (h*ng/mL) | 24610 | (24.2) | 243200 | (33.5) | 26120 | (41.9) |
| AUC_{0-∞} (h*ng/ml) | 49390 | (36.9) | 4881000 | (234) | 57290 | (57.7) |
| Cₘₐₓ (ng/mL) | 1913 | (29.2) | 11570 | (32.9) | 1903 | (23.5) |
| Tₘₐₓ (h) | 4.0 | (1.5-10.0) | 6.0 | (0.0-10.0) | 6.0 | (2.0-6.0) |

### Study Oteseconazole CL-011

A total of 217 subjects were randomized to receive oteseconazole and 109 subjects were randomized to receive placebo. All subjects were followed for 36 weeks after the end of treatment. Blood samples for determination of oteseconazole plasma concentrations were collected at Baseline, Day 14, Week 12, Week 24, Week 36, and Week 48 (End of Study (EOS]).

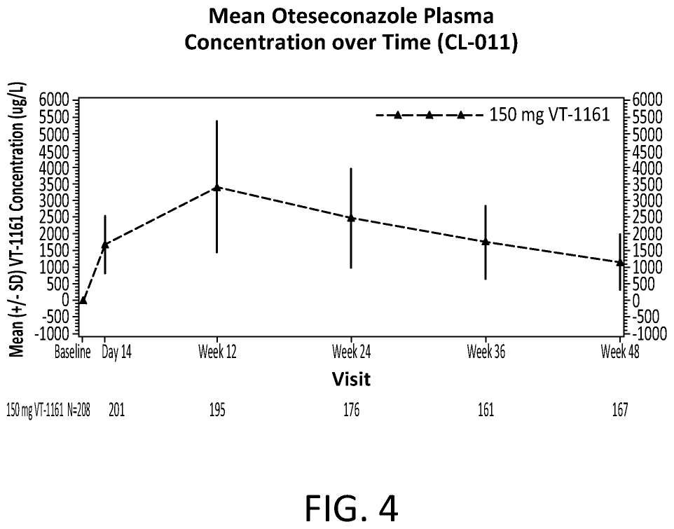

Oteseconazole mean (standard deviation [SD]) plasma concentration increased with dosing for 12 weeks reaching a maximum concentration of 3402.4 (1970.51) μg/L at Week 12. At the end of dosing, there was a slow decrease in oteseconazole plasma concentration, reaching 1148.5 (833.72) μg/L by Week 48 (FIG. 4).

Following the loading dose regimen of 150 mg oteseconazole QD for 7 days, the mean (SD) oteseconazole plasma concentration at Day 14 was 1676.2 (876.27) μg/L.

### Study Oteseconazole CL-012

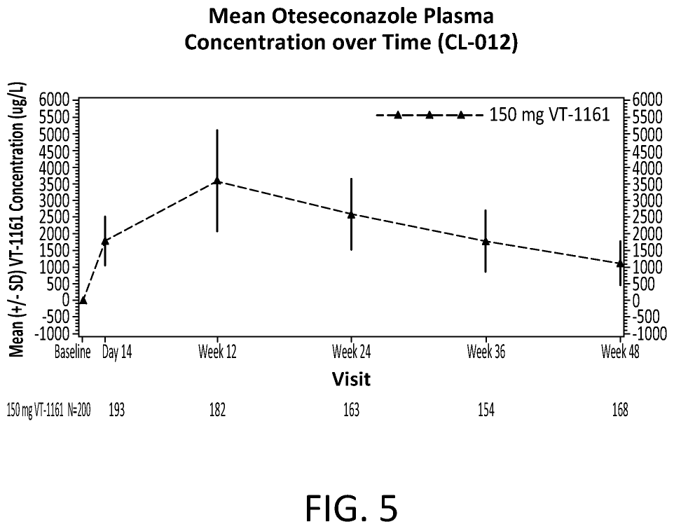

Oteseconazole mean (SD) plasma concentration increased with dosing for 12 weeks, reaching a maximum concentration of 3605.8 (1518.08) μg/L at Week 12. At the end of dosing, there was a slow decrease in oteseconazole plasma concentration, reaching 1122.0 (656.14) μg/L by Week 48 (FIG. 5).

Following the loading dose regimen of 150 mg oteseconazole QD for 7 days, the mean (SD) oteseconazole plasma concentration at Day 14 was 1785.5 (747.75) μg/L.

Oteseconazole administered as a 150 mg loading dose QD for 7 days, followed by a maintenance dose of 150 mg QW for 11 weeks resulted in plasma concentrations that provided efficacy consistent with the extent of efficacy in study CL-011 and the low dose regimen of study CL-006. The average percentage of subjects with one or more culture-verified acute VVC episodes in the 150 mg oteseconazole group was 3.9% versus 39.4% in the placebo group.

### Food Effect Study in Healthy Subjects (Oteseconazole CL-013)

Study Oteseconazole CL-013 (hereafter referenced as “CL-013”) was a single-center, Phase 1, open-label study in healthy adult female subjects to evaluate the effect of food on oteseconazole PK and to compare the bioavailability of oteseconazole capsule versus tablet when dosed with food. Administration of 150 mg oteseconazole (capsule formulation) following a high-fat and high-calorie meal increased mean Cₘₐₓ values and extent of exposure to oteseconazole area under the plasma concentration versus time curve from 0 to 72 hours (AUC₀₋₇₂) by approximately 45% and 36%, respectively, compared with fasting conditions. Administration of 150 mg oteseconazole (capsule formulation) following a low-fat and low-calorie meal increased Cₘₐₓ values by approximately 17% compared with fasting conditions, but yielded a comparable extent of exposure (AUC₀₋₇₂) relative to fasting conditions.

The extent of exposure was about 13% higher following administration of the capsule formulation compared with the tablet formulation, although both formulations yielded similar mean Cₘₐₓ values.

### Study Oteseconazole CL-017

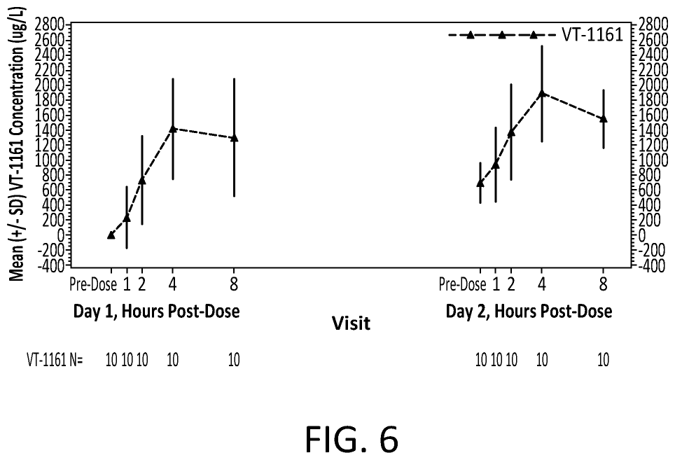

Oteseconazole mean (SD) plasma concentration during the intense PK sampling period on Day 1 peaked at 4 hours postdose (1425.8 [672.05] μg/L) and decreased slightly by 8 hours postdose (1301.06 [782.76] μg/L). On Day 2, mean (SD) oteseconazole plasma concentrations were 697.7 (264.40) μg/L at predose, then increased to 1895.0 (640.00) μg/L at 4 hours postdose, and decreased to 1555.6 (392.68) μg/L by 8 hours postdose (FIG. 6).

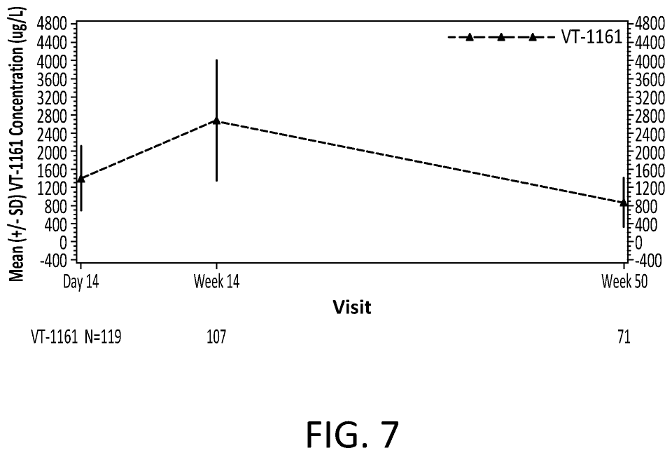

Over the period of Day 14 to Week 50, oteseconazole plasma concentrations were measured at Day 14, Week 14 (end of dosing), and Week 50 (end of follow-up period). Oteseconazole mean (SD) plasma concentration increased from 1396.9 (709.00) μg/L at Day 14 to 2679.3 (1318.94) μg/L at Week 14 before decreasing to 873.4 (534.49) μg/L at Week 50 (FIG. 7).

A summary of non-compartmental PK parameters of oteseconazole from the intense PK sampling over Day 1 and Day 2 is presented in Table 15.

On Day 1, geometric mean Cₘₐₓ was 1620 μg/L following oteseconazole 600 mg (4x 150 mg capsules) dosing. The Tₘₐₓ occurred at 4.01 hours; however, a wide range of Tₘₐₓ values were observed across subjects, ranging from 2.00 to 18.00 hours. Oteseconazole plasma concentrations remained elevated up to 24 hours postdose with a geometric mean last observed quantifiable concentration value of 651 μg/L. The AUC₀₋₂₄ was 20600 h*μg/L for oteseconazole 600 mg dose on Day 1 (Table 15).

Following the oteseconazole 600 mg dose on Day 1, plasma concentrations remained high and ranged from 354 to 1150 μg/L at 24 hours (predose Day 2) and contributed to trough and overall plasma concentrations on Day 2. The geometric mean Cₘₐₓ on Day 2 was 2010 μg/L following administration of oteseconazole 450 mg (3×150 mg capsules). Median Tₘₐₓ occurred at 4.00 hours on Day 2; however, Tₘₐₓ occurred in a narrower range (2.00 to 8.00 hours) compared with that observed on Day 1. The AUC₀₋₈ was 7570 h*μg/L and 11700 h*μg/L, for Day 1 and Day 2, respectively (Table 17).

TABLE 15
Oteseconazole Noncompartmental PK Parameters -
Day 1 and Day 2 (CL-017)

| | | | | | |
|---|---|---|---|---|---|
| Treatment, Day | Cₘₐₓ | Tₘₐₓ | AUC₀₋₈ | AUC₀₋₂₄ | Clast |
| Statistic | (μg/L) | (h) | (h*ng/L | (h*ng/L) | (μg/L) |

Oteseconazole 4 × 150 mg, Day 1

| | | | | | |
|---|---|---|---|---|---|
| N | 10 | 10 | 10 | 8 | 10 |
| Geometric | 1620 | NC | 7570 | 20600 | 651 |
| Mean | | | | | |
| Geo CV(%) | 42.2 | NC | 41.0 | 33.9 | 41.5 |
| Median ₐ | | 4.01 | | | |
| (min, max) | | (2.00, 18.00) | | | |

Oteseconazole 3 × 150 mg, Day 2

| | | | | | |
|---|---|---|---|---|---|
| N | 10 | 10 | 10 | NC | 10 |
| Geometric | 2010 | NC | 11700 | NC | 1510 |
| Mean | | | | | |
| Geo CV(%) | 27.7 | NC | 25.5 | NC | 28.2 |
| Median | | 4.00 | | | |
| (min, max) | | (2.00, 8.00) | | | |

ₐ Only presented for Tmax.

Oteseconazole administered as a 150 mg enhanced loading dose of 600 mg on Day 1 and 450 mg on Day 2 was noninferior to fluconazole administered as three 150 mg doses taken 72 hours apart (Days 1, 4, and 7). Furthermore, the enhanced loading dose, followed by maintenance doses of 150 mg QW for 11 weeks, resulted in plasma concentrations that provided efficacy consistent with the efficacy demonstrated in studies CL-011 and CL-012 and the low dose regimen of study CL-006. The average percentage of subjects with 1 or more culture-verified acute VVC episodes in the 150 mg oteseconazole group, including subjects who had a recurrence during the Induction Phase, was 5.1% versus 42.2% in the fluconazole/placebo group.

### EQUIVALENTS

Those skilled in the art will recognize, or be able to ascertain using no more than routine experimentation, many equivalents of the specific embodiments of the present application described herein. Such equivalents are intended to be encompassed by the following claims.

1. A pharmaceutical composition comprising an anhydrous polymorph of oteseconazole, a binder, a filler, a disintegrant and a surfactant and optionally a lubricant.

2. The composition of claim 1, wherein said composition is in a dosage unit.

3. The composition of claim 2, wherein said dosage unit is a tablet or a capsule.

4. The composition of claim 1 wherein the filler comprises lactose, microcrystalline cellulose or calcium hydrogen phosphate; the binder is selected from the group consisting of hydroxypropyl cellulose, pregelatinized maize starch, and polyvinylpyrrolidone; the disintegrant is selected from the group consisting of croscarmellose sodium, potato starch and sodium starch glycolate; and/or the surfactant is sodium lauryl sulfate.

5. The composition of claim 1 wherein the filler comprises silicified microcrystalline cellulose and lactose monohydrate, the binder is hydroxypropyl cellulose, the disintegrant is croscarmellose Sodium, and the surfactant is sodium lauryl sulfate.

6. The composition of claim 1, comprising about 150 mg oteseconazole, about 20 mg silicified microcrystalline cellulose, about 12 mg lactose monohydrate, about 6 mg hydroxypropyl cellulose, about 4 mg to about 8 mg croscarmellose sodium, about 2 mg sodium lauryl sulfate; and optionally about 2 mg magnesium stearate.

7. The composition of claim 1, comprising about 28% oteseconazole, about 20% silicified microcrystalline cellulose, about 42% lactose monohydrate, about 3% hydroxypropyl cellulose, about 2% to about 4% croscarmellose sodium, about 1% sodium lauryl sulfate; and optionally about 1% magnesium stearate.

8. (canceled)

9. The composition of claim 5, wherein the total weight of the dosage unit is between about 150 mg and about 250 mg.

10. (canceled)

11. The composition of claim 1, comprising about 300 mg oteseconazole, about 73 mg silicified microcrystalline cellulose, about 105 mg lactose monohydrate, about 16 mg hydroxypropyl cellulose, about 11 mg to about 22 mg croscarmellose sodium, about 5 mg sodium lauryl sulfate; and optionally about 5 mg magnesium stearate.

12. The composition of claim 11, comprising about 75% oteseconazole, about 10% silicified microcrystalline cellulose, about 6% lactose monohydrate, about 3% hydroxypropyl cellulose, about 2% to about 4% croscarmellose sodium, about 1% sodium lauryl sulfate; and optionally about 1% magnesium stearate.

13. The composition of claim 11, comprising about 57% oteseconazole, about 14% silicified microcrystalline cellulose, about 20% lactose monohydrate, about 3% hydroxypropyl cellulose, about 2% to about 4% croscarmellose sodium, about 1% sodium lauryl sulfate; and optionally about 1% magnesium stearate.

14. The composition of claim 11, comprising about 56% oteseconazole, about 14% silicified microcrystalline cellulose, about 20% lactose monohydrate, about 3% hydroxypropyl cellulose, about 2% to about 4% croscarmellose sodium, about 1% sodium lauryl sulfate; and optionally about 1% magnesium stearate.

15. The composition of claim 11, comprising about 67% oteseconazole, about 10% silicified microcrystalline cellulose, about 14% lactose monohydrate, about 3% hydroxypropyl cellulose, about 2% to about 4% croscarmellose sodium, about 1% sodium lauryl sulfate; and optionally about 1% magnesium stearate.

16. The composition of claim 11, wherein the total weight of the dosage unit is between about 400 mg and about 600 mg.

17. The composition of claim 1, wherein the total weight of the dosage unit is about 400 mg, about 440 mg, about 480 mg, about 520 mg, about 560 mg, or about 600 mg.

18. A method for treating or preventing vulvovaginal candidiasis comprising administering to a subject a composition of any of claims 1-17 in an amount effective to treat or prevent vulvovaginal candidiasis or RVVC.

19. A method for treating inflammatory bowel disease, psoriasis, systemic fungal infection, skin structure fungal infection, mucosal fungal infection, or onychomycosis comprising administering to a subject a composition of claim 1.

20. A method of manufacturing a composition of claim 1.

21. A method of treating or preventing vulvovaginal candidiasis in a subject in need thereof, comprising administering to the subject a composition of claim 1.

22. A method of treating inflammatory bowel disease, psoriasis, systemic fungal infection, skin structure fungal infection, mucosal fungal infection, or onychomycosis, in a subject in need thereof, comprising administering to the subject a composition of claim 1.
